(19) United States
(12) Patent Application Publication
(10) Pub. No.: US 2004/0167388 A1
(43) Pub. Date: Aug. 26, 2004

(54) Image detection system

(75) Inventor: Farhad A. Ghelmansarai, Danville, CA (US)

Correspondence Address: Attn: Elsa Keller, Legal Administrator, Siemens Corporation, Intellectual Property Department, 170 Wood Avenue South, Iselin, NJ, 08830, US

(73) Assignee: Siemens Medical Solutions USA, Inc.

(21) Appl. No.: 10/373,991

(22) Filed: Feb. 25, 2003

(51) Int. Cl. A61B 5/05; A61B 6/00

(52) U.S. Cl. 600/407; 600/427; 600/476

(57) ABSTRACT

A system includes activation of a local light emitter to emit local light, activation of a radiation emitter to emit radiation, and acquisition of an image based on the emitted radiation and on the local light. According to some embodiments, the emitted radiation is converted to second light, the local light and the second light are combined to create combined light, and the image is acquired based on the combined light.

### BACKGROUND

1. Field of the Invention

The present invention relates generally to radiation therapy, and more particularly to imaging systems used to detect therapeutic radiation.

2. Description

According to conventional radiation therapy, a radiation beam is directed toward a therapy target located within a patient. The radiation beam delivers a predetermined dose of therapeutic radiation to the target according to an established therapy plan. The delivered radiation kills cells within the target by causing ionizations within the cells.

A radiation therapy plan is designed to maximize radiation delivered to a target while minimizing radiation delivered to healthy tissue. These goals may not be achieved if the radiation is not delivered exactly as required by the therapy plan. More specifically, errors in radiation delivery can result in low irradiation of tumors and high irradiation of sensitive healthy tissue. The potential for mis-irradiation increases with increased delivery errors.

A portal imaging system may be used before and during therapy to verify that radiation is properly delivered to a therapy target. In practice, radiation is emitted from a radiation emitter toward a patient according to a therapy plan. An imaging device receives the radiation after it has passed through the patient and generates an image based on the received radiation. The image may be used to verify a location and a shape of the radiation field created by the emitted radiation. The image may also be used to verify radiation dosage in a case that an intensity of the emitted radiation corresponds to an intensity prescribed by the therapy plan.

If an operator wishes to verify only a location and a shape of the radiation field, the intensity of the emitted radiation may be significantly weaker then the intensity prescribed by the therapy plan. Such verification may advantageously reduce an amount of radiation delivered to healthy tissue. However, many conventional imaging devices are unable to produce useful images of weak radiation fields.

It would therefore be beneficial to provide a system that may offer more efficient and effective imaging of weak therapeutic radiation fields than previously available.

### SUMMARY

To address at least the above problems, some embodiments provide a system, method, apparatus, and means to activate a local light emitter to emit local light, to activate a radiation emitter to emit radiation, and to acquire an image based on the emitted radiation and on the local light. Some embodiments further provide conversion of the emitted radiation to second light, combination of the local light and the second light to create combined light, and acquisition of the image based on the combined light.

In some aspects, provided are a radiation emitter to emit radiation, a radiation converter to receive radiation from the radiation emitter and to emit first light based on the received radiation, a light emitter to emit second light, and an imaging device to acquire an image based on the first light and on the second light. Further to these aspects, also provided may be a combiner to receive the first light and the second light, and to emit combined light.

The claimed invention is not limited to the disclosed embodiments, however, as those of ordinary skill in the art can readily adapt the teachings herein to create other embodiments and applications.

### BRIEF DESCRIPTION OF THE DRAWINGS

Embodiments of the claimed invention will become readily apparent from consideration of the following specification as illustrated in the accompanying drawings, in which like reference numerals designate like parts, and wherein:

FIG. 1 is diagram illustrating a radiation therapy room according to some embodiments;

FIG. 2 is a diagram illustrating elements of an imaging device according to some embodiments;

FIG. 3 comprises a flow diagram of process steps according to some embodiments;

FIG. 4 is an image representing a radiation field;

FIG. 5 is an image of local light according to some embodiments; and

FIG. 6 is an image according to some embodiments.

### DETAILED DESCRIPTION

The following description is provided to enable any person of ordinary skill in the art to make and use embodiments of the claimed invention and sets forth the best modes contemplated by the inventor for carrying out the claimed invention. Various modifications, however, will remain readily apparent to those in the art.

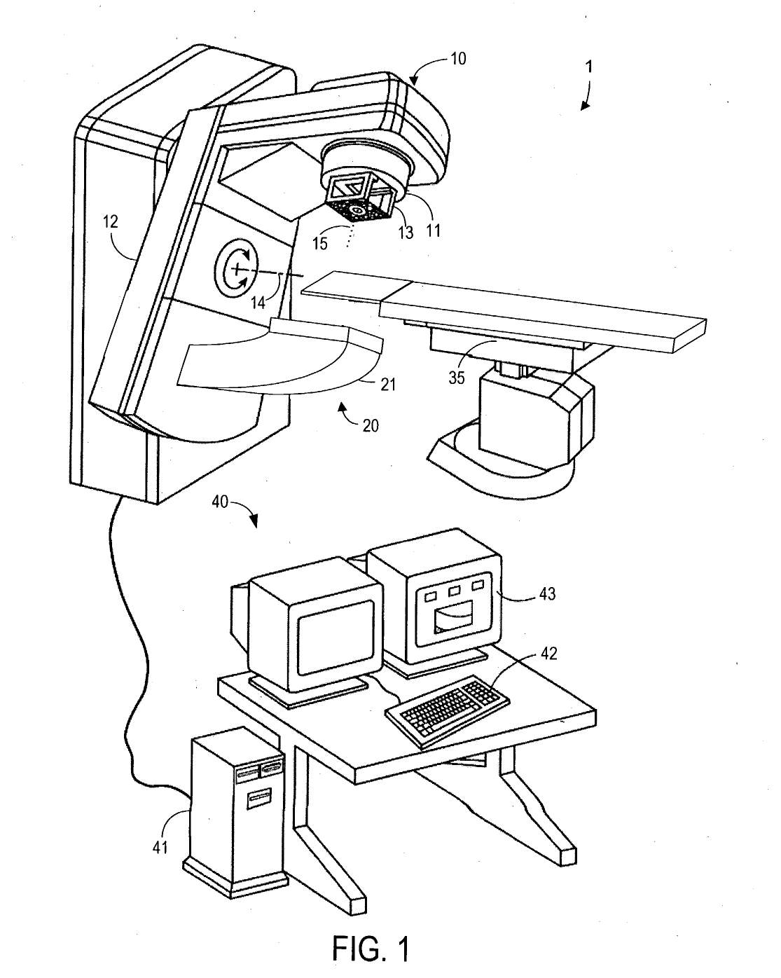

FIG. 1 illustrates radiation therapy room 1 pursuant to some embodiments. Radiation therapy room 1 includes linear accelerator (linac) 10, imaging device 20, table 35, and operator station 40. The elements of radiation therapy room 1 are primarily used to deliver therapeutic radiation to a patient according to a radiation therapy plan.

Linac 10 generates and emits the therapeutic radiation, and is primarily composed of treatment head 11 and gantry 12. Treatment head 11 includes a radiation emitter (not shown) for emitting a radiation beam used during calibration, beam verification, and/or therapy. The radiation beam may comprise electron, photon or any other type of radiation. Also included within treatment head 11 is a beam-shielding device, or collimator (not shown) for shaping the beam and for shielding sensitive surfaces from the beam.

Accessory tray 13 is mounted on treatment head 11 and may be configured to receive and securely hold attachments used during the course of planning and therapy. These attachments may include reticles, wedges, or the like for further defining radiation field sizes and intensities.

Treatment head 11 is fastened to a projection of gantry 12. Gantry 12 is rotatable around gantry axis 14 before, during and after radiation therapy. During such therapy, radiation is delivered from linac 10 to the radiation emitter of treatment head 11 and is emitted therefrom as a beam having axis 15. The beam is emitted towards a point, known as the isocenter, which is located at the intersection of axis 15 and gantry axis 14. Due to divergence of the radiation beam and the shaping of the beam by the aforementioned beam-shaping devices, the beam delivers radiation to a radiation field rather than only to the isocenter.

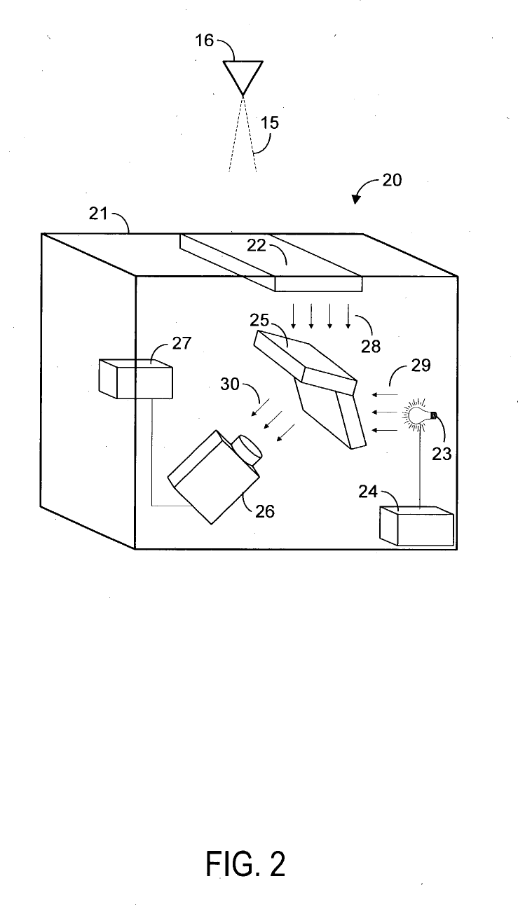

Imaging device 20 acquires images that are used before, during and after radiation therapy. For example, imaging device 20 is used to acquire images for verification and recordation of a patient position, of a radiation field, and of an internal patient portal to which radiation is delivered. Imaging device 20 may comprise a CCD or tube-based camera, or a flat-panel imaging device. Details of imaging device 20 according to some embodiments are presented below with respect to FIG. 2.

Table 35 supports a patient during radiation therapy. Table 35 is adjustable to ensure, along with rotation of gantry 12, that a therapy area of the patient is positioned at the isocenter. Table 35 may also be used to support phantoms, scanning ion chambers, and/or other devices used for calibration and/or verification.

Operator station 40 includes processor 41 in communication with an input device such as keyboard 42 and an operator console 43 (including one or more visual display units or monitors). Operator station 40 is typically operated by an operator who administers actual delivery of radiation therapy as prescribed by an oncologist. Operator station 40 may be located apart from linac 10, such as in a different room, in order to protect the operator from radiation. For example, linac 10 may be located in a heavily shielded room, such as a concrete vault, which shields the operator from radiation generated by linac 10. Operator station 40 may also be used to perform calibration and/or verification procedures prior to administering therapy.

Processor 41 may store processor-executable process steps according to some embodiments of the present invention. In some aspects, the process steps are executed by processor 41, linac 10, imaging device 20, and/or another device to activate a local light emitter to emit local light, to activate a radiation emitter to emit radiation, and to acquire an image based on the emitted radiation and on the local light. The process steps may additionally be executed to convert the emitted radiation to second light, combine the local light and the second light to create combined light, and acquire the image based on the combined light.

The above-described steps may also be embodied, in whole or in part, by hardware of processor 41, linac 10, and imaging device 20. Moreover, some embodiments may be embodied by hardware and/or software of a standalone device connected between imaging device 20 and operator station 40, between linac 10 and imaging device 20, or elsewhere.

Of course, radiation therapy room 1 may include less or more elements than those shown. Accordingly, embodiments are not limited to the illustrated elements.

FIG. 2 is a diagram illustrating elements of imaging device 20 according to some embodiments. As shown, imaging device 20 comprises light-proof housing 21, which in turn comprises scintillator 22, local light emitter 23, light emitter control 24, optical combiner 25, camera 26, and frame grabber 27. The individual elements of imaging device 20 may be implemented by any suitable device or devices.

In some embodiments, scintillator 22 is a gadolinium-sulfide layer that absorbs x-ray radiation and emits visible photons having an intensity proportional to that of the absorbed x-rays. Scintillator 22 may also comprise a Cesium-iodide or other type of scintillator. Light emitter 23 may comprise a light bulb, light-emitting diode, or other device for emitting light that is detectable by camera 26. Optical combiner 25 may comprise a prism or other device for combining two light sources to create combined light. Additionally, camera 26 may comprise an array of charge-coupled devices or a vacuum tube.

In operation, scintillator 22 receives radiation beam 15 from radiation emitter 16 of treatment head 11 and emits light 28 in proportion to the intensity of the received radiation. According to some embodiments, one or more objects such as table 35, a patient, and a phantom are disposed between radiation emitter 16 and scintillator 22. Portions of radiation beam 15, and therefore portions of resulting light 28, may be attenuated by the objects. Consequently, images that are acquired based on light 28 may present features of the objects.

Light emitter 23 emits light 29 in response to a signal received from light emitter control 24. In this regard, the term “light” is used herein to describe visible radiation emitted by scintillator 22 and by light emitter 23, while the term “radiation” is used to identify radiation emitted by radiation emitter 16 of treatment head 11.

Optical combiner 25 receives light 28 and light 29 and combines the received light to create combined light 30. Camera 26 receives combined light 30 and records the intensity of combined light 30 as stored electrical charge. Frame grabber 27 reads a video signal output by camera 26 in real-time and produces still frame images therefrom. Still frame images may be output from imaging device 20 to acquisition and image processing software executed by processor 41 or by another device and may alternatively or additionally be output to an operator through console 43.

Imaging device 20 may include less or more elements than shown in FIG. 2. For example, mirrors and/or lenses may be used to manipulate the light emitted by scintillator 22, light emitter 23, and/or optical combiner 25. Moreover, the physical dimensions of the elements and the spatial relationships of the elements to one another are not limited to the dimensions and relationships shown in FIG. 2.

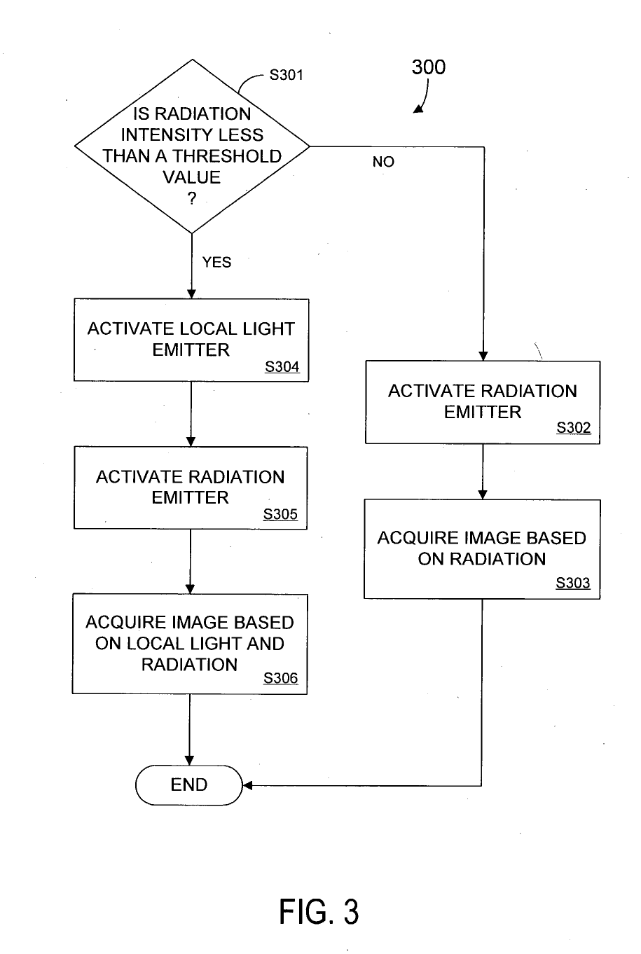

FIG. 3 comprises a flow diagram of process steps 300 according to some embodiments of the invention. Process steps 300 may be embodied by hardware and/or software of processor 41, linac 10, imaging device 20, and/or another device in direct or indirect communication with imaging device 20.

Process steps 300 begin at step S301, in which it is determined whether an intensity of radiation to be delivered to a patient is greater than a threshold value. The threshold value may represent a minimum value required to acquire a satisfactory image using imaging device 20. The threshold value may differ depending upon the particular embodiment of imaging device 20, the type of radiation, and/or dimensions of the therapeutic radiation field.

In one example of step S301, processor 41 may analyze a therapy plan according to which radiation will be delivered in order to determine whether the intensity of the radiation is greater than the threshold value. According to some embodiments, an operator operates station 40 in order to indicate whether the intensity of the radiation is greater than the threshold value. Step S301 may occur any time prior to delivery of the radiation.

Flow proceeds to step S302 if the determination in step S301 is negative. In step S302, radiation emitter 16 is activated to emit radiation according to the therapy plan. The radiation is received by scintillator 22, which emits light 28 toward optical combiner 25 as described above. Since substantially no light is received by optical combiner 25 from light emitter 23, combined light 30 from optical combiner 25 is substantially identical to light 28.

Combined light 30 is received by camera 26 in order to acquire an image in step S303. The acquired image represents a radiation field produced by radiation beam 15 at the surface of scintillator 28. The radiation field is also affected by attenuative properties of any patient structures located between radiation emitter 16 and scintillator 22. The latter phenomena causes the image to present patient structures that are intercepted by radiation beam 15, and thereby allows verification of a location of the radiation field.

If the determination in step S301 is positive, local light emitter 23 is activated in step S304. Local light emitter 28 thereafter emits local light 29. Step S304 may comprise transmission of a local light enable signal from light control 24 to light emitter 23. Local light emitter 23 may provide one or more light intensities and/or wavelengths. In such embodiments, the local light enable signal may indicate an intensity and/or wavelength of light to be emitted. The intensity and/or wavelength of local light 29 may be controlled based on an intensity of radiation beam 15, an intensity of light 28, characteristics of optical combiner 25, characteristics of camera 26, and/or other factors.

Radiation emitter 16 is then activated in step S305 to emit radiation beam 15 according to the therapy plan. As described above, scintillator 22 receives radiation beam 15 and emits light 28 in proportion to the intensity of the received radiation. Optical combiner 25 receives light 28 and light 29 and combines the received light to create combined light 30. Next, in step S306, camera 26 receives combined light 30 and acquires an image based on combined light 30 as stored electrical charge. Frame grabber 27 may also read a video signal output by camera 26 in real-time and produce still frame images therefrom.

In some embodiments, local light 29 causes imaging device 20 to operate in a shot noise limited condition. Shot noise is noise caused by random fluctuations in the motion of charge carriers in a conductor. Shot noise arises from the statistical nature of photon emission and detection.

The intensity of local light 28 may therefore be increased until the shot noise of local light 28 dominates other noise sources of imaging device 20, such as CCD circuit noise (dark current, fixed pattern noise, etc.), thermal noise, and output amplifier noise. This procedure is equivalent to setting the other noise sources to zero, and may significantly increase the signal-to-noise ratio. More particularly, the signal-to-noise ratio in a shot noise limited condition is proportional to the square root of the number of measured photons and the bandwidth of imaging system 20. As a result, the signal-to-noise ratio in a shot noise limited condition is maximized for a given beam 15, scintillator 22, light emitter 23, optical combiner 25, camera 26, and system bandwidth.

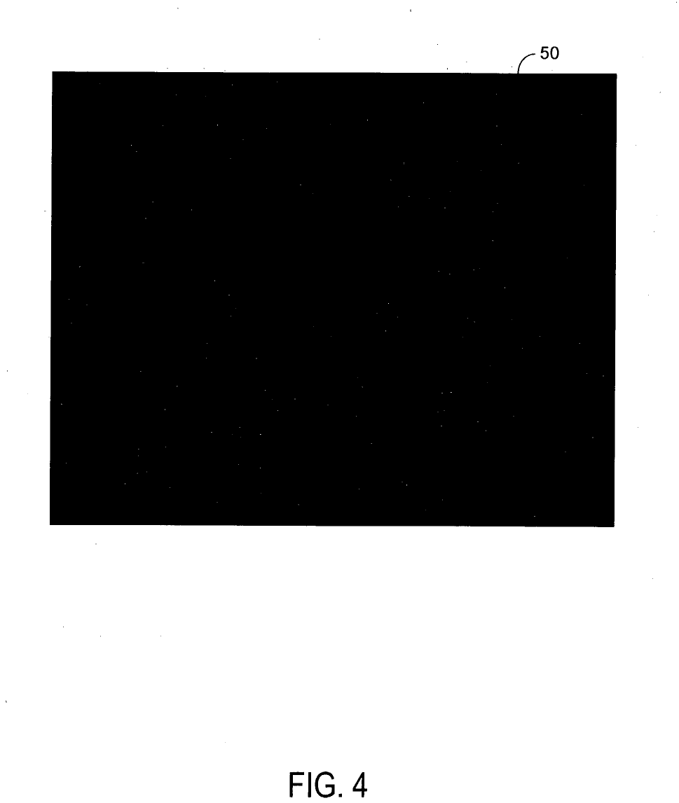

FIGS. 4 through 6 will be used to explain the effects of a shot noise limited condition according to some embodiments. FIG. 4 shows image 50 acquired by imaging device 20 without the use of local light emitter 23. Image 50 represents a phantom placed on table 35 at the isocenter of linac 10. The phantom is irradiated by a low-intensity radiation field produced by beam 15. Due to the low intensity, features of the phantom cannot be discerned from image 50. More specifically, the signal level of light emitted by scintillator 22 in response to the low-intensity radiation field is less than a noise level of camera 26. The small white specks within image 50 are caused by scattered x-rays that reach the imaging elements of camera 26.

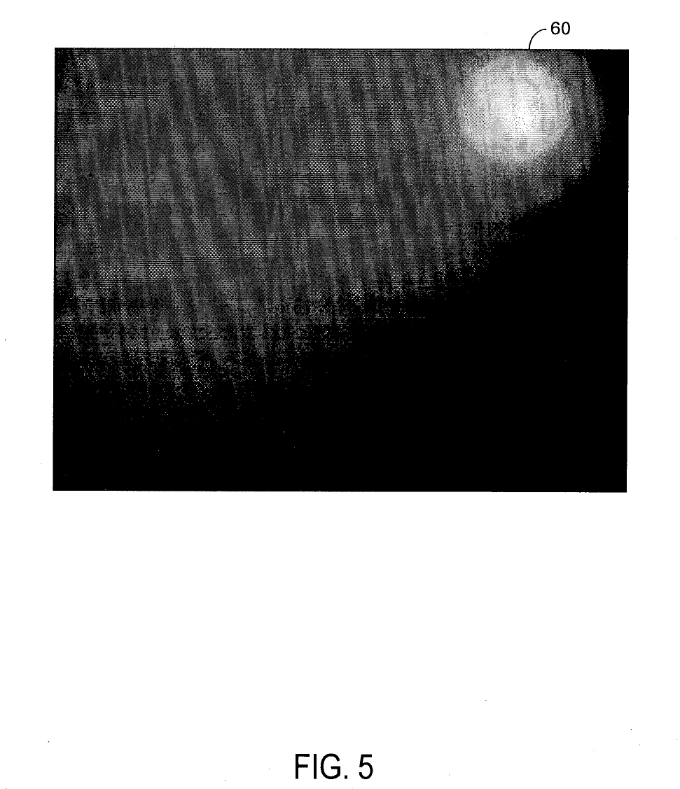

Image 60 of FIG. 5 is acquired by imaging device 20 while light 29 is emitted by local light emitter 23 and while radiation emitter 16 of treatment head 11 is not activated. The phantom may or may not be placed on table 35 during acquisition of image 60. Since scintillator 22 receives no radiation from radiation emitter 16, combined light 30 emitted by optical combiner 25 substantially represents only light 29. In the example of FIG. 5, local light emitter 23 is a flashlight. As described above, the presence of light 29 may cause imaging device 20 to operate in a shot noise limited condition.

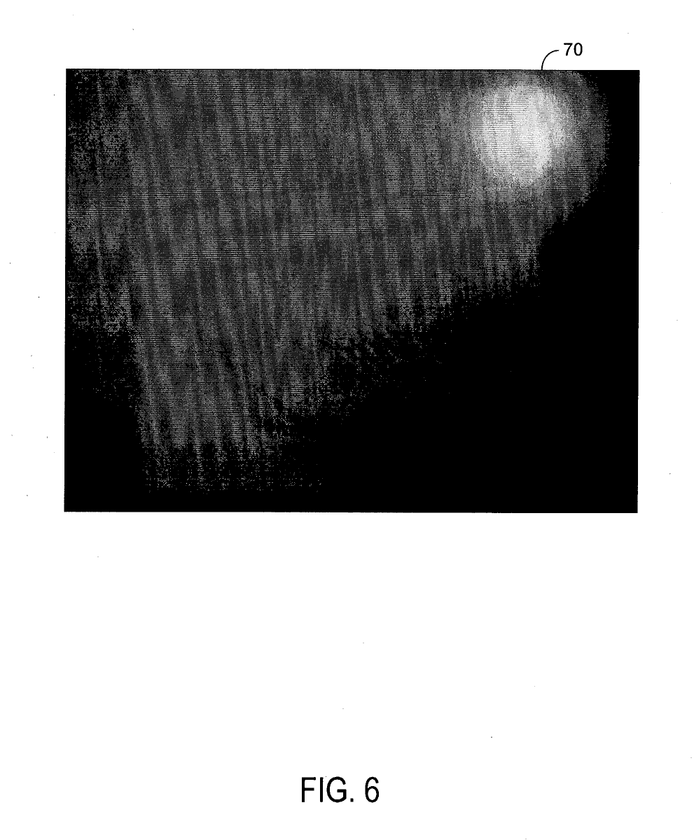

FIG. 6 shows image 70 of the phantom acquired while imaging device 20 is in a shot noise limited condition. In particular, local light emitter 23 is activated and emits light 29. Radiation emitter 16 of treatment head 11 emits radiation having a same intensity as the radiation represented in image 50. As shown, features of the phantom can be discerned in image 70 more clearly than in image 50, even though both images were acquired using a same low-intensity radiation emitted from radiation emitter 16. This increase in clarity from image 50 to image 70 corresponds to an increase in signal-to-noise ratio that is due to the shot noise limited condition of imaging device 20. Again, the shot noise limited condition results from the dominance of the shot noise of local light 28 over other noise sources.

Those in the art will appreciate that various adaptations and modifications of the above-described embodiments can be configured without departing from the scope and spirit of the claimed invention. For example, embodiments may differ from process steps 300. Moreover, embodiments may relate to the imaging of any visible light, regardless of source. Therefore, it is to be understood that, within the scope of the appended claims, the claimed invention may be practiced other than as specifically described herein.

What is claimed is:

1. A method comprising:
activating a local light emitter to emit local light;
activating a radiation emitter to emit radiation; and
acquiring an image based on the emitted radiation and on the local light.

2. A method according to claim 1, further comprising:
determining that an intensity of the radiation is lower than a threshold value,
wherein the local light emitter is activated if the intensity of the radiation is lower than the threshold value.

3. A method according to claim 1, further comprising:
converting the emitted radiation to second light.

4. A method according to claim 3, further comprising:
combining the local light and the second light to create combined light.

5. A method according to claim 4, wherein the acquiring step comprises:
acquiring the image based on the combined light.

6. A computer-readable medium storing computer-executable process steps, the process steps comprising:
a step to activate a local light emitter to emit local light;
a step to activate a radiation emitter to emit radiation; and
a step to acquire an image based on the emitted radiation and on the local light.

7. A medium according to claim 6, the process steps further comprising:
a step to determine that an intensity of the radiation is lower than a threshold value,
wherein the local light emitter is activated if the intensity of the radiation is lower than the threshold value.

8. A medium according to claim 6, wherein the acquiring step comprises:
a step to acquire the image based on combined light, the combined light comprising the local light and light representing the emitted radiation.

9. An apparatus to emit radiation, to emit first light based on the received radiation, to emit second light, and to acquire an image based on the first light and on the second light.

10. An apparatus according to claim 9, the apparatus to determine that an intensity of the radiation is lower than a threshold value, and to activate the local light emitter if the intensity of the radiation is lower than the threshold value.

11. An apparatus according to claim 9, the apparatus to convert the emitted radiation to second light.

12. An apparatus according to claim 11, the apparatus to combine the local light and the second light to create combined light.

13. An apparatus according to claim 12, the apparatus to acquire the image based on the combined light.

14. A system comprising:
a radiation emitter to emit radiation;
a radiation converter to receive radiation from the radiation emitter and to emit first light based on the received radiation;
a light emitter to emit second light; and
an imaging device to acquire an image based on the first light and on the second light.

15. A system according to claim 14, further comprising:
a combiner to receive the first light and the second light, and to emit combined light,
wherein the imaging device is to acquire the image based on the combined light.

16. A system according to claim 14, wherein the converter is a scintillator.

17. A system according to claim 14, further comprising:
a light-proof housing,
wherein the converter, the light emitter and the imaging device are disposed within the light-proof housing.

18. A system according to claim 14, further comprising:
a light emitter control to activate the light emitter.

19. A system according to claim 18, wherein the light control is to activate the light emitter based on an indication that an intensity of the radiation is lower than a threshold value.

20. A system according to claim 18, wherein the light control is to activate the light emitter based on a local light enable signal.

21. A system according to claim 14, wherein the second light causes the imaging device to operate in a shot noise-limited condition.

22. A method comprising:
receiving light from a light source;
activating a local light emitter to emit local light; and
acquiring an image based on the received light and on the local light.

23. A method according to claim 22, further comprising:
determining that an intensity of the received light is lower than a threshold value,
wherein the local light emitter is activated if the intensity of the received light is lower than the threshold value.
